(19) United States
(12) Patent Application Publication
(10) Pub. No.: US 2004/0082558 A1
(43) Pub. Date: Apr. 29, 2004

(54) Administration of estradiol metabolites for inhibition of drug-induced nephrotoxicity

(76) Inventors: Stevan P. Tofovic, Pittsburgh, PA (US); Edwin K. Jackson, Pittsburgh, PA (US); Raghvendra K. Dubey, Glenshaw, PA (US)

Correspondence Address: Mary-Elizabeth Buckles, REED SMITH LLP, Suite 1100-East Tower, 1301 K Street, N.W., Washington, DC, 20005-3317, US

(21) Appl. No.: 10/633,410

(22) Filed: Aug. 4, 2003

Related U.S. Application Data
(60) Provisional application No. 60/400,060, filed on Aug. 2, 2002.

(51) Int. Cl. A61K 31/56

(52) U.S. Cl. 514/182

(57) ABSTRACT

Methods and composition are provided for the treatment or prevention of drug-induced nephrotoxicity and related conditions. More particularly, this invention relates to compositions comprising estradiol metabolites, and prodrugs thereof, that may be incorporated in various controlled release formulations.

### RELATED APPLICATIONS

This application claims priority from U.S. Provisional Application No. 60/400,060 filed Aug. 2, 2002.

### FIELD OF THE INVENTION

The present invention relates to methods and compositions using estradiol metabolites which may be incorporated into drug delivery compositions. More particularly, the present invention relates to the use of estradiol metabolites for the prevention or for the treatment of drug-induced nephrotoxicity. The estradiol metabolites include 2-hydroxyestradiol, 4-hydroxyestradiol, 2-methoxyestradiol, and 4-methoxyestradiol, and prodrugs thereof.

### BACKGROUND OF THE INVENTION

Chronic renal disease is characterized by accelerated arteriosclerosis and an excessive rate of cardiovascular morbidity and mortality. Linder, A. B., et al., N. Engl. J. Med. 290:697, (1974), the disclosure of which is incorporated herein by reference. Recent data indicate the progression of chronic renal disease occurs more rapidly and more frequently in men than in women and appears to occur independently of blood pressure or serum cholesterol levels. United States Renal Data System:1993 Annual Data Report, MD. U.S. Department of Health and Human Services, Public Health Services, National Institutes of Health, p. xvi (1993); Neugarten, J., et al., J. Am. Soc. Nephrol. 11:319 (2000); and Silbiger, S. R., et al., Am. J. Kid. Disease 25:515 (1995), the disclosures of which are incorporated herein by reference. The resistance of kidneys in women to the progression of renal disease has been frequently attributed to 17β-estradiol, the main estrogenic steroid produced by the human ovary, Silbiger, S. R., et al., Am. J. Kid. Disease 25:515 (1995), the disclosure of which is incorporated herein by reference. However, the effects of compounds such as 17β-estradiol on drug-induced nephrotoxicity are not known. In addition, because estrogenic activity of estradiol increases the risk of cancer in women, Manson, J. E., et al., N. Engl. J. Med. 345: 34 (2001), the disclosure of which is incorporated herein by reference, and is feminizing in men, Brawnstein, G. D., New England J. Med. 328:490 (1993), the disclosure of which is incorporated herein by reference, it would be of limited value as a treatment for nephrotoxicity.

The present invention is directed to alternative approaches for the treatment or prevention of drug-induced nephrotoxicity. One aspect of the present invention is the use of metabolites of 17β-estradiol in this capacity. Such metabolites have a structure similar to 17β-estradiol but can be administered in a dose that has minimal estrogenic effects. Ball, P., et al., Am. J. Obstet. & Gyn. 51:611 (1980), the disclosure of which is incorporated herein by reference.

Other features and advantages of the invention will be apparent from the following description of the preferred embodiment, and from the claims.

Citation of the above-referenced documents is not intended as an admission that any of the foregoing is prior art. All statements as to the date or representation as to the contents of the documents is based on subjective characterization of information available to the applicants and does not constitute any admission as to the accuracy of the dates or contents of these documents. The disclosures of all documents cited herein are hereby incorporated herein by reference.

### SUMMARY OF THE INVENTION

Methods are provided for preventing or treating drug-induced nephrotoxicity and related conditions in an individual. These treatments may be used in either gender because of their lack of a feminizing estrogenic effect.

These and other objects of the invention are achieved by one or more of the following embodiments.

In one aspect, the invention features a method for preventing or treating drug-induced nephrotoxicity in an individual, comprising administering to the individual a therapeutically effective amount of a composition comprising an estradiol metabolite.

In a preferred embodiment, the estradiol metabolite is selected from the group consisting of 2-methoxyestradiol, 4-methoxyestradiol, 2-hydroxyestradiol, and 4-hydroxyestradiol.

In another preferred embodiment the composition is a prodrug of the estradiol metabolite.

In a further preferred embodiment, the composition comprises a controlled release formulation.

In another aspect, the invention features a method for preventing or treating drug-induced proteinuria in an individual, comprising administering to the individual a therapeutically effective amount of a composition comprising an estradiol metabolite.

In another aspect, the invention features a method for preventing or treating drug-induced decreases in glomerular filtration rate in an individual, comprising administering to the individual a therapeutically effective amount of a composition comprising an estradiol metabolite.

In a further aspect, the invention features a method for preventing or treating drug-induced infiltration of inflammatory cells into renal tissue in an individual, comprising administering to the individual a therapeutically effective amount of a composition comprising an estradiol metabolite.

In yet another aspect, the invention features a method for preventing or treating drug-induced excessive proliferation of renal cells in an individual, comprising administering to the individual a therapeutically effective amount of a composition comprising an estradiol metabolite.

In yet another aspect, the invention features a method for preventing or treating drug-induced excessive extracellular matrix protein production in renal tissue in an individual, comprising administering to the individual a therapeutically effective amount of a composition comprising an estradiol metabolite.

Other features and advantages of the invention will be apparent from the following description of the preferred embodiment, and from the claims.

### DESCRIPTION OF THE DRAWINGS

The following figures form part of the present specification and are included to further demonstrate certain embodiments. These embodiments may be better understood by reference to one or more of these drawings in combination with the detailed description of specific embodiments presented herein.

FIGS. 1A-1C are graphs showing: exemplary plasma creatinine (FIG. 1A), percentage change in creatinine clearance (FIG. 1B), and absolute creatinine clearance (FIG. 1C) in control animals and in puromycin aminonucleoside-treated rats (PAN) receiving either vehicle or 2-hydroxyestradiol (2OHE). Results represent mean ±SEM. (2F-Anova, 2-factor analysis of variance; a, p<0.05 for Control vs. PAN and PAN+2OHE; b, p<0.05 for PAN vs. PAN+2OHE; c, p<0.05, for PAN vs. Control and PAN+2OHE).

FIGS. 2A and 2B are graphs showing: exemplary urinary protein excretion (UPE) (FIG. 2A) and urinary protein excretion divided by creatinine clearance in control animals and in puromycin aminonucleoside-treated rats (PAN) receiving either vehicle or 2-hydroxyestradiol (2OHE). Results represent mean ±SEM. (UPE/CrCl, UPE divided by creatinine clearance; 2F-Anova, 2-factor analysis of variance; a, p<0.05 for Control vs. PAN and PAN+2OHE; b, p<0.05 for PAN vs. PAN+2OHE).

FIG. 3 is a graph showing exemplary assessment of glomerular immunohistochemical staining for proliferating cell nuclear antigen (PCNA) in control animals (clear bar) and in puromycin aminonucleoside-treated rats (PAN) receiving either vehicle (solid bar) or 2-hydroxyestradiol (2OHE) (hatched bar). Staining was assessed by quantitative image analysis with a SAMBA 4000 image analyzer. Results represent mean ±SEM of the labeling index. (1F-Anova, 1-factor analysis of variance).

FIG. 4 is a graph showing assessment of glomerular immunohistochemical staining for ED1 positive cells in glomeruli (right) and interstitium (left) of control rats (clear bar), rats treated with puromycin aminonucleoside (PAN) (solid bar) and rats treated with PAN and 2-hydroxyestradiol (2OHE) (hatched bar). Quantitative image analysis with a SAMBA 4000 image analyzer was utilized to quantify staining. Results represent mean ±SEM. (2F-Anova, 2-factor analysis of variance).

FIG. 5A-5C are photographs showing representative collagen IV immunochemistry of cortical sections from a puromycin aminonucleoside-treated rat (FIG. 5A), a puromycin aminonucleoside-treated rat also treated with 2-hydroxyestradiol (FIG. 5B) and a control rat at a magnification of 400× (FIG. 5C).

FIG. 6 is a graph showing exemplary assessment of glomerular immunohistochemical staining for collagen IV of control rats (clear bar), puromycin aminonucleoside-treated rats (PAN) (solid bar), and PAN-treated rats also treated with 2-hydroxyestradiol (2OHE) (hatched bar). Staining was assessed by quantitative image analysis with a SAMBA 4000 image analyzer. Results represent the mean ±SEM of the labeling index. (1F-Anova, 1-factor analysis of variance)

### DETAILED DESCRIPTION OF THE INVENTION

### I. Definitions

The term “estradiol metabolite(s)” refers to metabolites of 17β-estradiol such as catecholestradiols and methoxyestradiols which exert little estrogenic activity and have a low affinity for the estrogen receptor, examples of which include 2-methoxyestradiol, 4-methoxyestradiol, 2-hydroxyestradiol and 4-hydroxyestradiol.

The term “individual” refers to either a human or animal of the male or female gender.

“Biodegradable” refers to polymers that dissolve or degrade in vivo within a period of time that is acceptable in a particular therapeutic situation. This time is less than five years and typically less than one year after exposure to a physiological pH and temperature, such as a pH ranging from 6 to 9 and a temperature ranging from 25° C. to 40° C.

“PAN” refers to puromycin aminonucleoside.

“2OHE” or “2-OHE” refers to 2-hydroxyestradiol.

The term “prodrug” refers to a compound that is metabolized or converted to become an active drug in the body.

“PCNA” refers to proliferating cell nuclear antigen.

“UPE” refers to urinary protein excretion.

“2F-Anova” refers to 2-factor analysis of variance and “1F-Anova” refers to 1-factor analysis of variance.

According to the present invention, an isolated or biologically pure estradiol metabolite is a compound that has been removed from its natural milieu. As such, “isolated” and “biologically pure” do not necessarily reflect the extent to which the compound has been purified. An isolated compound of the present invention can be obtained from its natural source or can be produced by chemical synthesis.

### II. METHODS

The present invention provides compositions and methods for the prevention or treatment of nephrotoxicity. In particular, the present invention is directed to the prevention or treatment of nephrotoxicity and related conditions that are drug-induced. The invention further provides controlled release formulations of the present compositions such as biodegradable microparticles or nanoparticles, patches, crystals, gels, hydrogels, liposomes, implants or vaginal rings associated with the compositions and methods of the present invention.

Examples of drugs that may cause drug-induced nephrotoxicity in accordance with the present invention include puromycin aminonucleoside (PAN); aminoglycosides, such as gentamicin; cephalosporins, such as cephaloridine; calcineurin inhibitors, such as tacrolimus or sirolimus; and radiographic contrast agents, such as diatrizoate, iothalamate or metrizoate salts. Drug-induced nephrotoxicity may also be caused by non-steroidal anti-inflammatories, anti-retrovirals, anti-cytokines, immunosuppressants, oncological drugs or ACE inhibitors. The drug-induced nephrotoxicity may further be caused by amphotericin B, analgesic abuse, ciprofloxacin, clopidogrel, cocaine, cox-2 inhibitors, diuretics, foscamet, gold, ifosfamide, immunoglobin, Chinese herbs, interferon, lithium, mannitol, mesalamine, mitomycin, nitrosureas, penicillamine, penicillins, pentamidine, quinine, rifampin, streptozocin, sulfonamides, ticlopidine, triamterene, valproic acid, doxorubicin, glycerol, cidofovir, tobramycin, neomycin sulfate, colistimethate, vancomycin, amikacin, cefotaxime, cisplatin, acyclovir, lithium, interleukin-2, cyclosporin or indinavir.

Estradiol metabolites that may be used in the compositions and methods of the present invention include one or more of 2-methoxyestradiol, 2-hydroxyestradiol, 4-methoxyestradiol and 4-hydroxyestradiol and prodrugs of 2-hydroxyestradiol, 4-hydroxyestradiol, 2-methoxyestradiol and 4-methoxyestradiol. Other compounds that may be used in the present invention include those which are hydroxylated or methylated at other sites as is known in the art.

Therapeutic compositions of the present invention can be formulated in an excipient that the individual to be treated can tolerate. Examples of such excipients include water, saline, Ringer's solution, dextrose solution, Hank's solution, and other physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability.

Examples of buffers for use in formulations for the therapeutic compositions of the present invention include phosphate buffer, bicarbonate buffer and Tris buffer, while examples of preservatives include thimerosal, cresols, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline is added prior to administration.

The present invention also has the objective of providing suitable topical, oral, systemic and parenteral formulations of the pharmaceutical compounds herein provided. The formulations can be administered in a wide variety of therapeutic dosage forms in conventional vehicles for administration. For example, the compounds can be formulated for oral administration in the form of tablets, capsules (each including timed release and sustained release formulations), pills, powders, granules, elixirs, tinctures, solutions, suspensions, syrups and emulsions. Likewise, they may also be administered intravenously (both bolus and infusion), during angioplasty/catheterization, intraperitoneally, subcutaneously, topically with or without occlusion, or intramuscularly, all using formulations well known to those of ordinary skill in the pharmaceutical arts.

Therapeutic compositions according to the present invention include a carrier. Carriers include compounds that increase the half-life of a therapeutic composition in the treated individual.

Suitable carriers include, but are not limited to, polymeric controlled release vehicles, biodegradable implants, liposomes, bacteria, viruses, other cells and glycols.

Biodegradable microparticles or nanoparticles used in the present invention may have a composition that includes one or more polymers such as poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and polyorthoester, biodegradable polyurethanes, blends and copolymers thereof.

The preferred methods of administration of the compositions of the present invention include subcutaneous administration and intramuscular administration. Additional methods of administration include, but are not limited to, parenteral administration, intradermal administration, buccal administration, transdermal administration, transmucosal administration, intravaginal administration, intranasal administration, suppositories, or oral administration. Such methods of administration are well known in the art.

### EXAMPLES

The following examples illustrate the manner in which the present invention may be practiced. It is understood, however, that the techniques described in the examples which follow are for the purposes of illustration only, and thus can be considered to only constitute preferred modes for the practice of the present invention and the invention is not to be regarded as limited to any of the specific methods or materials herein. Variations can be made by those of skill in the art without departing from the spirit and scope of the invention.

### Materials

### Baseline Measurements

A total of thirty-five, male Sprague Dawley rats (approximately 290 grams) were used. Rats were housed in the University of Pittsburgh Medical Center animal care facility (temperature, 22° C.; light cycle, 12 hours; relative humidity, 55%). Animals were fed Pro Lab RMH 3000 rodent diet (PMI Nutrition Inc., St Louis, Mo.) and were given water ad libitum. Institutional guidelines for animal welfare were followed, and the University of Pittsburgh Institutional Animal Care and Use Committee approved experimental protocols.

Before initiating treatment, and 3, and 6 and 11 weeks into the treatments, animals were placed in metabolic cages and allowed to acclimatize for two days before conducting the 24-hour measurements of urine volume, food and water intake and urinary sodium, potassium, creatinine, and protein excretion. Tail vein blood samples were also taken for measurement of plasma sodium, potassium, albumin and creatinine. Plasma and urine samples were analyzed for sodium and potassium concentrations using a flame photometer (Model IL-943; Instrumentations Laboratory Inc., Lexington, Ma.) and for creatinine concentrations using a creatinine analyzer (Creatinine Analyzer 2, Beckman Instrument, Inc., Fullerton Calif., USA). Total urine proteins were measured by a spectrophotometric assay using bicinchoninic acid reagent (Pierce; Rockford, Ill.) and a modification of Lowry method. Lowry, O. H., et al., J. Biol. Chem. 193: 265 (1951), the disclosure of which is incorporated herein by reference.

### Experimental Design

After baseline metabolic parameters were measured, animals were randomly assigned to receive subcutaneous saline (3 ml/kg; Control group, n=9) or puromycin aminonucleoside (PAN) (75 mg/kg; drug-induced nephrotoxic animals, n=26). PAN is a drug widely used to produce drug-induced nephrotoxicity in laboratory animals. Grond, J., et al., Contributions to Nephrology 60:83 (1988), the disclosure of which is incorporated herein by reference. Injections of PAN (20 mg/kg) were repeated after 2, 4, 8 and 10 weeks of treatment. Three hours after the initial injections, control animals were implanted with osmotic minipumps (model 2ML4, Alza, Palo Alto, Calif.) containing vehicle (polyethylene glycol 400, 2.5 μl/hour), whereas PAN-treated animals were implanted with osmotic minipumps containing either vehicle (PAN group, n=13) or 2-hydroxyestradiol (10 μg/kg/hour, PAN+2OHE group, n=13). Assignment to the PAN and PAN+2OHE groups was random. Six animals in the PAN group and 2 animals in the PAN+2OHE group died during the treatment, and these animals were not used in the final data analysis.

At 12 weeks into treatment, animals were anesthetized with pentobarbital (45 mg/kg i.p.) and instrumented for measurements of renal hemodynamics and excretory function. Two polyethylene (PE)-50 catheters were inserted into left jugular vein for delivery of supplemental anesthetics and saline infusion (50 μl/min), respectively. Another PE-50 catheter was inserted into left carotid artery and connected to a blood-pressure analyzer (Micro-Med., Inc., Louisville, Ky.) for continuous measurement of blood pressure and heart rate. A PE-10 catheter was inserted into the left ureter to facilitate collection of urine, and a flow probe (Model 1RB; Transonic Systems, Inc., Ithaca, N.Y.) was placed on the left renal artery for determination of renal blood flow, which was used to calculate renal vascular resistance. Next, an infusion of ¹⁴C-inulin (0.035 μCi/20 μl saline/min) was initiated and after 60 minutes, two 30-minute clearance periods were conducted. A mid-point blood sample (300 μl) was collected and plasma and urine ¹⁴C-inulin radioactivity was measured. Renal clearance of ¹⁴C-inulin was calculated as an estimate of glomerular filtration rate.

An aliquot of mid-point blood sample drawn during the first clearance period was used for testosterone measurements. Plasma testosterone levels were determined by radioimmunoassay using a commercial kit provided by ICN Biomedicals, Inc. (Costa Mesa, Calif.) according to the protocol of the manufacturer.

Animals were killed by an overdose of anesthetic and kidneys were removed and weighed. One kidney was fixed in 10% formalin buffer and the second kidney was quick-frozen in liquid nitrogen and stored at −70° C. Kidney tissue samples were sectioned and processed into paraffin blocks for light microscopy. For labeling of collagen IV, renal cortical segments (5 μm) were incubated overnight at 4° C. with rabbit anti-mouse collagen IV antibody (dilution 1/500 ) obtained from Chemicon International Inc. (Temecula, Calif.). A primary monoclonal mouse antibody (1/200 dilution; Dako Corporation, Carpenteria, Calif.) was used to label proliferating cell nuclear antigen (PCNA). A rat ED1 antibody (Serotec, Raleigh, N.C.) specific for a monocyte/macrophage cytoplasmatic antigen was used to label glomerular and interstitial macrophages. Nonspecific staining was assessed by replacing the primary antibody with phosphate buffered saline. Sections were washed and further developed according to the directions of the manufacturer (Dako Corporation) using the LSAB2 kit that contained a second antibody linked to avidin and peroxidase conjugated biotin. Immunochemical staining for collagen IV and PCNA quantitatively was done with a SAMBA 4000 image analyzer (Image Products International, Chantilly, Va.) using specialized computer software (Immuno-Analysis, version 4.1, Microsoft, Richmond, Wash.), a color video camera and a Compaq computer. Software designed for immunostaining analysis enabled the operator to set density threshold values by averaging several fields on the negative control tissues in which the primary antibody was replaced with phosphate buffered saline. Background subtraction was then performed automatically on every tissue. Ten high power fields (×400) were assessed for staining density or positively marked cells for ED-1. The results are reported as the labeling index, which represents the percentage of the total examined area that stained positively. Staining intensity of positive areas was also assessed (mean optical density) and a mean quick score was then calculated (mean optical density×labeling index).

### Data Analysis

All data are presented as mean ±S.E.M. Statistical analyses were performed using the Number Cruncher Statistical Software program (Kaysville, Utah). Group comparison for data from metabolic studies (repeated measurements) were performed by one-(1F) or two-(2F) hierarchical analysis of variance (Anova) as appropriate, followed by Fisher's LSD test for post-hoc comparisons. Comparison of data from acute experiments and from histological analysis (single point data) was performed by 1F- Anova (all three groups) or Student's t-test (PAN versus PAN+2OHE). The probability value of p<0.05 was considered statistically significant.

### II. Results

Chronic administration of PAN resulted in severe renal damage. PAN induced severe proteinuria, increased plasma creatinine levels and reduced creatinine clearance. PAN-treated animals also had reduced body weight and increased food and fluid intake, urine output and fractional excretion of sodium and potassium as shown in Table 1 below.

TABLE 1
Metabolic parameters in Control and PAN-nephropathic rats receiving vehicle (PAN) or 2-OHE (PAN + 2OHE)

| | | Weeks of Treatment | | | | 2F-Anova |
|---|---|---|---|---|---|---|
| Parameter | | 0 wks | 3 wks | 6 wks | 11 wks | treatment effect |
| Body Weight (g) | Control | 287 ± 3 | 343 ± 5 | 382 ± 6 | 421 ± 5 | a: p < 0.001 |
| | PAN | 293 ± 4 | 313 ± 1* | 337 ± 15 | 382 ± 27* | |
| | PAN + 2OHE | 291 ± 5 | 281 ± 5* [symbol] | 330 ± 8* [symbol] | 317 ± 13* [symbol] | b: p < 0.05 |
| Food Intake (g/kg/day) | Control | 80 ± 3 | 60 ± 2 | 60 ± 7 | 43 ± 3 | a: p < 0.05 |
| | PAN | 75 ± 2 | 74 ± 4 | 69 ± 3 | 53 ± 5 | |
| | PAN + 2OHE | 76 ± 2 | 75 ± 4 | 60 ± 5 | 58 ± 6 | |
| Fluid Intake (ml/kg/day) | Control | 141 ± 6 | 107 ± 3 | 95 ± 7 | 85 ± 8 | a: p < 0.001 |
| | PAN | 128 ± 9 | 182 ± 16* | 166 ± 13* | 159 ± 17* | |
| | PAN + 2OHE | 129 ± 6 | 169 ± 16* | 142 ± 12* | 147 ± 14* | |
| Urine Volume (ml/kg/day) | Control | 41.9 ± 3.7 | 39.4 ± 4.2 | 39.5 ± 5.3 | 28.9 ± 2.4 | a: p < 0.001 |
| | PAN | 42.7 ± 6.6 | 100.6 ± 11.1 | 93.7 ± 12.3 | 113.1 ± 16.6* | |
| | PAN + 2OHE | 40.3 ± 4.0 | 86.9 ± 7.0 | 71.8 ± 7.0 | 82.6 ± 7.5* | |
| Sodium Excretion | Control | 4.20 ± 0.49 | 3.42 ± 0.32 | 3.12 ± 0.20 | 2.47 ± 0.33 | c: <0.03 |
| (mEq/day/kg) | PAN | 4.48 ± 0.95 | 3.80 ± 0.58 | 3.92 ± 0.38 | 4.18 ± 0.58* | |
| | PAN + 2OHE | 3.35 ± 0.18 | 5.76 ± 0.57* | 4.21 ± 0.43 | 3.12 ± 0.66 | |
| Potassium Excretion | Control | 9.76 ± 0.80 | 9.52 ± 0.61 | 8.05 ± 0.66 | 6.88 ± 0.77 | |
| (mEq/day/kg) | PAN | 10.9 ± 0.72 | 10.7 ± 0.82 | 8.39 ± 0.61 | 9.63 ± 1.60 | |
| | PAN + 2OHE | 7.54 ± 0.52 | 12.3 ± 0.61 | 9.17 ± 0.63 | 8.36 ± 1.25 | |
| Creatinine Excretion | Control | 14.7 ± 1.8 | 12.3 ± 0.5 | 12.6 ± 0.6 | 15.8 ± 1.4 | a: p < 0.001 |
| (mg/day) | PAN | 14.9 ± 2.5 | 11.1 ± 1.1 | 8.8 ± 0.9* | 18.7 ± 3.0 | |
| | PAN + 2OHE | 13.0 ± 1.0 | 10.9 ± 0.7 | 9.8 ± 0.7* | 12.8 ± 2.8 | |
| FE - Na+ (%) | Control | 0.19 ± 0.04 | 0.20 ± 0.02 | 0.22 ± 0.02 | 0.24 ± 0.02 | |
| | PAN | 0.13 ± 0.01 | 0.37 ± 0.06 | 0.71 ± 0.13* | 1.18 ± 0.35* | b: p < 0.001 |
| | PAN + 2OHE | 0.18 ± 0.03 | 0.50 ± 0.10 | 0.34 ± 0.04 | 0.37 ± 0.04 | d: p < 0.001 |
| FE - K+ (%) | Control | 13.0 ± 2.1 | 11.4 ± 0.9 | 12.1 ± 1.3 | 18.5 ± 1.5 | a: p < 0.001 |
| | PAN | 10.5 ± 0.7 | 27.3 ± 3.7* | 30.1 ± 4.6* | 47.1 ± 7.7* | |
| | PAN + 2OHE | 11.6 ± 1.4 | 32.1 ± 4.4* | 17.3 ± 2.5* | 29.7 ± 1.7* | b: p < 0.001 |
| Plasma Triglycerides (mg/ml) | Control | 115 ± 26 | 78 ± 19 | 119 ± 25 | 96 ± 16 | a: p < 0.001 |
| | PAN | 117 ± 15 | 465 ± 90* | 531 ± 139* | 722 ± 247* | |
| | PAN + 2OHE | 119 ± 17 | 552 ± 127* | 413 ± 56* | 809 ± 130* | |
| Plasma Cholesterol (mg/dL) | Control | 53.8 ± 4.1 | 46 ± 3.1 | 51.1 ± 4.4 | 47.6 ± 4.2 | a: p < 0.001 |
| | PAN | 53.6 ± 2.3 | 353 ± 39.4* | 283.5 ± 39.5* | 349.6 ± 43.0* | |
| | PAN + 2OHE | 51.2 ± 2.2 | 315 ± 39.3* | 248.3 ± 19.3* | 340.6 ± 27.3* | |

2F-Anova (treatment effect): a - Control vs. nephropathic animals; b - PAN vs. PAN + 2OHE; c - Cont vs PAN + 2OHE; d - Cont vs PAN Fisher's LSD “t” test: * vs Control group; [symbol] - vs PAN group. FE - Na+ and FE - K+ indicate fractional excretion of sodium and potassium, respectively.

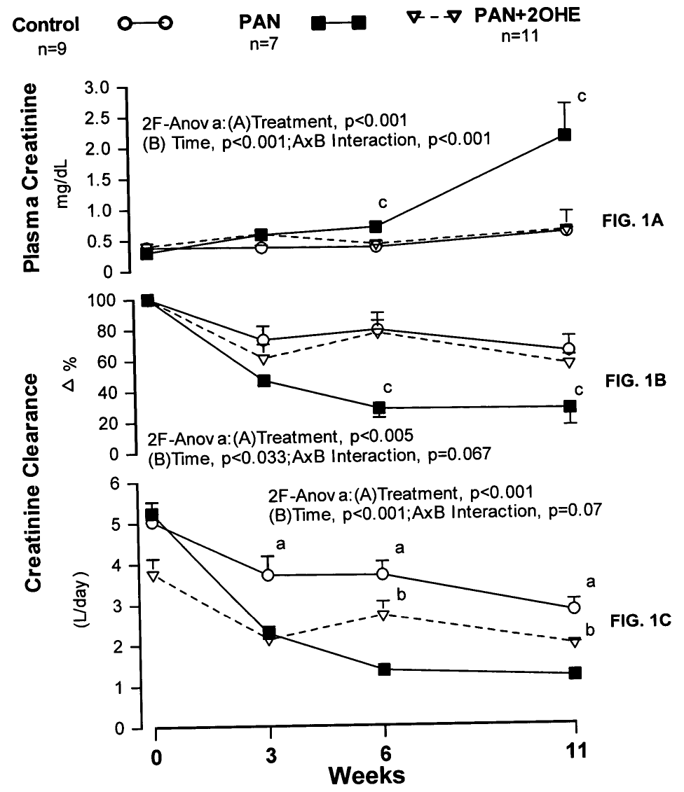

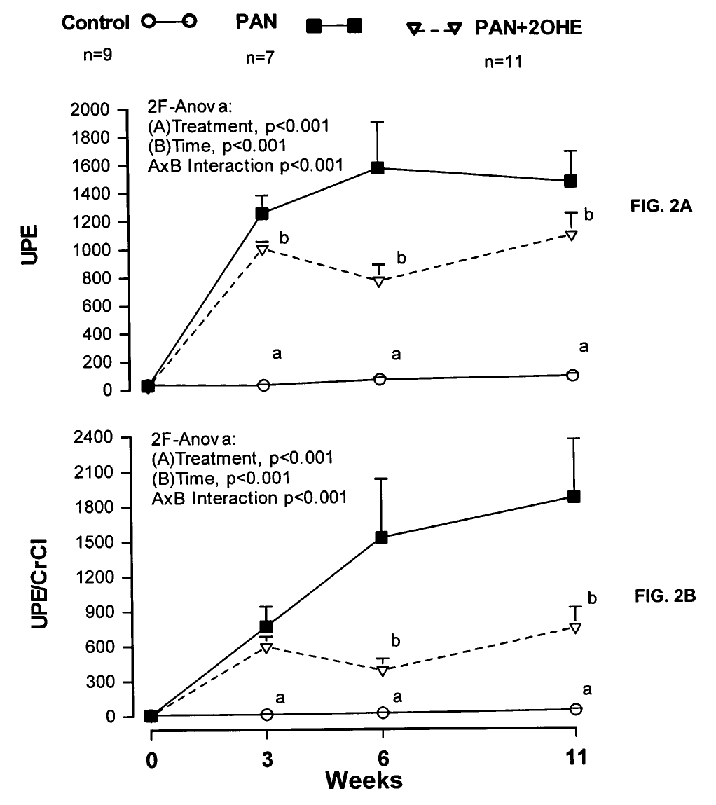

Chronic treatment with 2-OHE attenuated PAN-induced increases in plasma creatinine levels (FIG. 1A) and attenuated the decline in creatinine clearance caused by PAN (FIGS. 1B and 1C). Furthermore, 2-OHE reduced PAN-induced increases in urinary protein excretion (UPE, FIG. 2A) and this effect became even more significant when UPE was corrected for reduction in glomerular excretory function (i.e., creatinine clearance, FIG. 2B). Importantly, 2-OHE reduced PAN-induced mortality by 66%.

Acute measurements of renal hemodynamics and excretory function revealed increased blood pressure and renal vascular resistance and decreased renal blood flow, renal plasma flow, hematocrit and glomerular filtration rate (GFR, inulin clearance) in PAN-treated rats as compared with control rats as shown in Table 2 below. Importantly, 2-OHE abolished PAN-induced changes in blood pressure and significantly attenuated PAN-induced changes in GFR.

TABLE 2
Acute measurements of renal hemodynamic and excretory function in
control animals (Control) and in PAN-nephropathic animals receiving
vehicle (PAN) or 2-hydroxyestradiol (PAN + 2OHE) for 12 weeks

| PARAMETERS | | | |
|---|---|---|---|
| (M ± SE) | CONTROL | PAN | PAN + 2OHE |
| Body weight | 433 ± 5^{a} | 368 ± 15 | 353 ± 15 |
| Left Kidney | 1.44 ± 0.2^{a} | 2.10 ± 0.13 | 2.34 ± 0.09 |
| (g) | | | |
| Mean Blood | 133.4 ± 2.3 | 153.0 ± 6.1 | 138.2 ± 4^{b} |
| Pressure (mm Hg) | | | |
| Renal Blood Flow | 5.03 ± 028^{a} | 2.73 ± 0.24 | 2.80 ± 0.18 |
| (ml/min/g kidney) | | | |
| Hematocrit (%) | 50 ± 1^{a} | 27 ± 2 | 30 ± 3 |
| Renal Plasma Flow | 2.5 ± 0.12 | 2.00 ± 0.19 | 1.96 ± 0.12 |
| (ml/min/g kidney) | | | |
| Renal Vascular | 28.3 ± 1.26^{a} | 63.6 ± 7.6 | 58.5 ± 4.5 |
| Resistance (mm Hg/ | | | |
| ml/min/g kid) | | | |
| Urine Volume | 5.7 ± 0.6 | 4.43 ± 0.85 | 3.33 ± 0.26 |
| (μl/min/g kidney) | | | |
| Glomerular Filtra- | 1.65 ± 0.12^{a} | 0.21 ± 0.07 | 0.54 ± 0.06^{b} |
| tion Rate (ml/min/g | | | |
| kidney) | | | |

2F-Anova, p < 0.05;
^{a}Control vs Nephropathic animals;
^{b}PAN vs PAN + 2OHE

PAN-treated animals tended to have lower testosterone levels as compared with control rats with intact kidneys; however, treatment with 2-OHE did not lower testosterone levels (1.49±0.2, 0.92±0.27, and 0.91±0.22 ng/ml, Control, PAN and PAN+2OHE groups, respectively). These findings rule out the possibility that 2-OHE prevents drug induced nephrotoxicity via estrogenic effects to reduce androgen levels. Reckelhoff, J. F., et al., Clin. & Exp. Pharm. & Phys. 26:127 (1999), the disclosure of which is incorporated herein by reference.

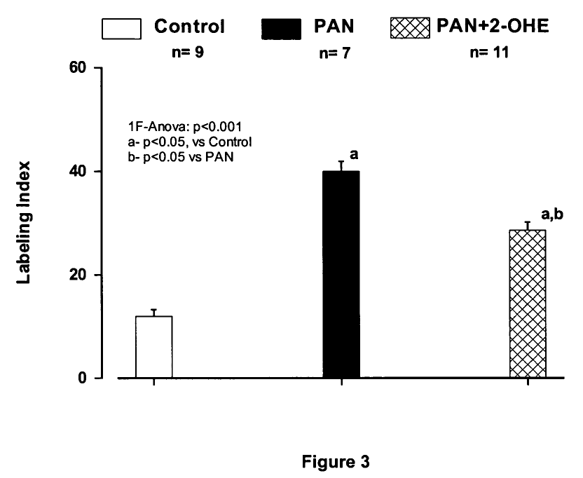

Immunohistochemical stains for collagen IV, PCNA and ED1 positive cells were significantly higher in PAN-treated rats versus control animals with intact kidneys. Assessment of glomerular staining for PCNA by quantitative image analysis is shown in FIG. 3. As assessed by quantitative analysis, the area positive for PCNA staining (labeling index, FIG. 3) was markedly expanded in glomeruli from PAN-treated rats compared with control animals with intact kidneys. Treatment with 2-OHE significantly reduced the labeling index of glomerular immunoreactive PCNA in PAN-treated animals. These results indicate that PAN induces proliferation of glomerular mesangial cells and that this effect of PAN is inhibited by 2-OHE.

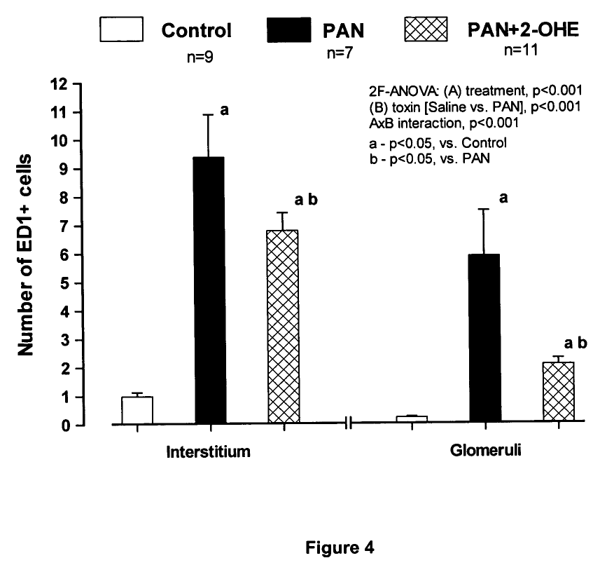

FIG. 4 illustrates the influence of 2-OHE on glomerular and interstitial macrophage infiltration. PAN induced a several-fold increase in the number of ED1 positive cells, a reliable marker of macrophage infiltration, both in glomeruli and in the renal interstitium. Importantly, treatment with 2-OHE significantly (P<0.001) reduced the number of inflammatory cells in glomeruli and in the interstitium of PAN-treated kidneys as shown in FIG. 4. These results indicate that PAN induces glomerular and interstitial infiltration of inflammatory cells and this infiltration is attenuated by 2-OHE.

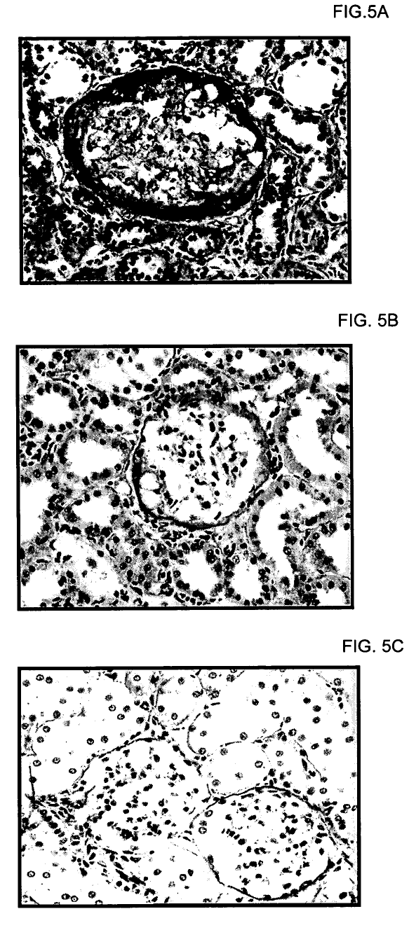

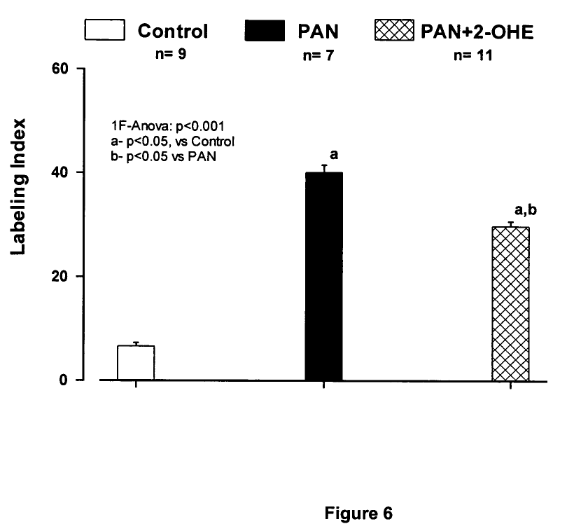

Representative examples of glomerular staining for collagen IV in cortical glomeruli from each study group are shown in FIGS. 5A-5C. A significant increase in glomerular collagen IV content was detected in PAN-treated rats (FIG. 5A) as compared with control animals with intact kidneys (FIG. 5C). Treatment with 2-OHE significantly decreased PAN-induced increases in collagen IV (FIG. 5B) and significantly reduced the labeling index of glomerular immunoreactive collagen IV in PAN-treated animals (FIG. 6). These results indicate that PAN expands the extracellular matrix in the glomerular and that this adverse effect of PAN is attenuated by 2-OHE.

While the disclosed methods and compositions have been described in terms of the specific embodiments of the invention, it will be apparent to those of ordinary skill in the art that variations may be applied without departing from the concept, spirit and scope of the claimed invention. More specifically, it will be apparent that certain agents that are both chemically and physiologically related may be substituted for the agents described herein while the same or similar results would be achieved. All such similar substitutes and modifications apparent to those skilled in the art are deemed to be within the invention as defined by the appended claims. Accordingly, it is understood that the drawings and the descriptions herein are proffered only to facilitate comprehension of the invention and should not be construed to limit the scope hereof.

We claim:

1. A method for preventing or treating drug-induced nephrotoxicity in an individual, comprising:
administering to said individual a therapeutically effective amount of a composition comprising an estradiol metabolite.

2. The method of claim 1, wherein said estradiol metabolite is selected from the group consisting of 2-hydroxyestradiol, 2-methoxyestradiol, 4-hydroxyestradiol and 4-methoxyestradiol.

3. The method of claim 1, wherein said composition comprises a controlled release formulation of said composition.

4. The method of claim 1, wherein said composition comprises a prodrug of said estradiol metabolite.

5. A method for preventing or treating drug-induced proteinuria in an individual, comprising:
administering to said individual a therapeutically effective amount of a composition comprising an estradiol metabolite.

6. The method of claim 5, wherein said estradiol metabolite is selected from the group consisting of 2-hydroxyestradiol, 2-methoxyestradiol, 4-hydroxyestradiol and 4-methoxyestradiol.

7. The method of claim 5, wherein said composition comprises a controlled release formulation of said composition.

8. The method of claim 5, wherein said composition comprises a prodrug of said estradiol metabolite.

9. A method for preventing or treating drug-induced decreases in glomerlular filtration rate in an individual, comprising:
administering to said individual a therapeutically effective amount of a composition comprising an estradiol metabolite.

10. The method of claim 9, wherein said estradiol metabolite is selected from the group consisting of 2-hydroxyestradiol, 2-methoxyestradiol, 4-hydroxyestradiol and 4-methoxyestradiol.

11. The method of claim 9, wherein said composition comprises a controlled release formulation of said comoposition.

12. The method of claim 9, wherein said composition comprises a prodrug of said estradiol metabolite.

13. A method for preventing or treating drug-induced infiltration of inflammatory cells into renal tissue in an individual, comprising:
administering to said individual a therapeutically effective amount of a composition comprising an estradiol metabolite.

14. The method of claim 13, wherein said estradiol metabolite is selected from the group consisting of 2-hydroxyestradiol, 2-methoxyestradiol, 4-hydroxyestradiol and 4-methoxyestradiol.

15. The method of claim 13, wherein said composition comprises a controlled release formulation of said composition.

16. The method of claim 13, wherein said composition comprises a prodrug of said estradiol metabolite.

17. A method for preventing or treating drug-induced excessive proliferation of renal cells in an individual, comprising:
administering to said individual a therapeutically effective amount of a composition comprising an estradiol metabolite.

18. The method of claim 17, wherein said estradiol metabolite is selected from the group consisting of 2-hydroxyestradiol, 2-methoxyestradiol, 4-hydroxyestradiol and 4-methoxyestradiol.

19. The method of claim 17, wherein said composition comprises a controlled release formulation of said composition.

20. The method of claim 17, wherein said composition comprises a prodrug of said estradiol metabolite.

21. A method for preventing or treating drug-induced excessive extracellular matrix protein production in renal tissue in an individual, comprising:
administering to said individual a therapeutically effective amount of a composition comprising an estradiol metabolite.

22. The method of claim 21, wherein said estradiol metabolite is selected from the group consisting of 2-hydroxyestradiol, 2-methoxyestradiol, 4-hydroxyestradiol and 4-methoxyestradiol.

23. The method of claim 21, wherein said composition comprises a controlled release formulation of said composition.

24. The method of claim 21, wherein said composition comprises a prodrug of said estradiol metabolite.
